(19) United States
(12) Patent Application Publication
(10) Pub. No.: US 2016/0009755 A1
(43) Pub. Date: Jan. 14, 2016

(54) CYRRHETINIC ALKYL ESTERS AND PROTECTED DERIVATIVES THEREOF

(71) Applicant: REVLON CONSUMER PRODUCTS CORPORATION, New York, NY (US)

(72) Inventors: Harry Cai, Skillman, NJ (US); Dariush Hosseinpour, Mason, OH (US); Alan Meyers, Flanders, NJ (US)

(21) Appl. No.: 14/771,687

(22) PCT Filed: Feb. 28, 2014
(86) PCT No.: PCT/US14/19327

§ 371 Date: Aug. 31, 2015

Related U.S. Application Data
(60) Provisional application No. 61/771,368, filed on Mar. 1, 2013.
(60) Provisional application No. 61/885,058, filed on Oct. 1, 2013.

(51) Int. Cl. C07J 63/00

(52) U.S. Cl. CPC C07J 63/008

(57) ABSTRACT

The present invention is directed to cyrrhetinic alkyl ester compounds, a method of making the compounds, cosmetic compositions containing the compound(s) and methods of using the same for the treatment of inflammation in human skin. The compounds and cosmetic compositions containing thereof provide various advantageous properties to the human skin.

### BACKGROUND OF THE INVENTION

1. Field of the Invention

The present invention relates to cyrrhetinic alkyl ester compounds, methods of making the compounds, cosmetic compositions containing the compounds and methods of using the same for the treatment of inflammation in human skin. The compounds and the cosmetic compositions containing such compounds provide various advantageous properties to the human skin (including hair, nail and lip). The compounds and compositions may have, among others, moisturizing and/or softening properties and would be useful for treating and/or relieving mild to moderate dry skin. The compounds and compositions may also have collagen-production inducing and skin-lightening properties. The compounds and compositions may also have an anti-inflammatory effect on human skin. The compounds and compositions may also have a correcting and perfecting benefit to sensitive skin.

2. Related Background Art

For most, if not all, having beautiful skin is very important. There are, however, challenges in achieving and maintaining beautiful skin. One of the many challenges is the exposure to the environments, e.g., sun radiation, dryness of the air, chemical (natural and/or artificial) exposure that causes damage, etc. Another challenge is aging. Both exposure to the environments and aging may cause the skin to be dry, sensitive, wrinkled or to have lines, and to lose its elasticity; they may also cause darkening/discoloration of the skin and the degradation of collagen fibers therein.

Dry skin is generally characterized by cracking, flaking, or scaling of the skin of the hands, feet, neck, face, or other parts of the body. Dry skin may result from a hereditary disorder known as ichthyosis which is a severe form of dry skin. The more common form of dry skin is a mild to moderate form of dry skin which arises due to exposure to environmental conditions of low humidity in the fall and winter seasons of the temperate climate zones. These environmental conditions give rise to, in skin areas exposed thereto, a loss of moisture from such skin areas, resulting in the formation of fissures, chaps, cracks, or flakes in the affected skin areas.

Various compounds have been proposed for use in treating or relieving dry skin. These compounds are generally formulated with other materials for topical use in the form of a lotion, cream, or ointment.

Aging causes, among others, the degradation of collagen fibers in the skin. The degradation results in the skin being flaccid and lacking firmness. Aging, generally in combination with exposure to the sun, also causes discoloration/darkening of the skin. Attempts have been made to delay, or even reverse, the effects of aging by delaying the degradation and/or increasing collagen production; or by lightening or evening out the discoloration of the skin.

There is a need for a compound that can treat and/or relieve dry skin, eczema, and restore the properties of the skin barrier. There is also a need for a compound that can delay and/or prevent the effects of aging. In addition, there is a need for a compound with anti-inflammatory properties. Further, there is a need for a stable cosmetic composition containing such a compound that provides the benefits associated with the compound.

### SUMMARY OF THE INVENTION

The present invention is directed to cyrrhetinic alkyl ester compounds having the following formula (I):

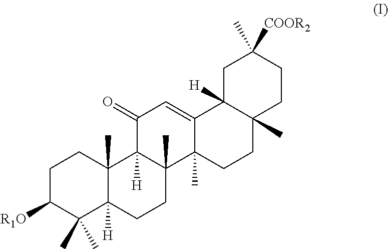

wherein R₁ is selected from the group consisting of H- and a protecting group and R₂ is a substituted or unsubstituted moiety selected from the group consisting of C1 to C6 alkyl, C2 to C6 alkenyl, C2 to C6 alkynyl,

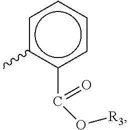

aryl, and heteroaryl, wherein R₃ is C1 to C6 alkyl, and the cosmetically acceptable salts thereof. The protecting group may be selected from those well-known in the art for the protection of alcohol groups and include, without limitation, those from protecting groups such as acetyl, benzoyl, benzyl, beta-methoxymethyl ether, methoxymethyl ether, p-methoxy benzyl ether, methylthiomethyl ether, pivaloyl, tetrahydropyranal, trityl and silyl ether. An example of a suitable protecting group is an acetyl group.

Exemplary cosmetically acceptable salts include, without limitation, hydrochloric, malic, lactic, acetic and citric salts. The compounds of this invention have chiral centers and thus may exist in enantiomeric form. The compounds of this invention include all racemates as well as all enantiomeric forms.

The present invention includes the method of making the cyrrhetinic alkyl ester compounds of formula (I) and the cosmetically acceptable salts thereof. The method of making the cyrrhetinic alkyl ester compounds of the invention comprises the step of esterification of a glycyrrhetinic acid compound having the formula (III).

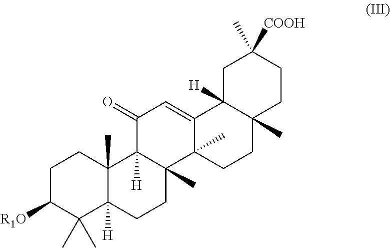

with a substituted or unsubstituted alcohol selected from the group consisting of C1-C6 alkyl alcohol, C2-C6 alkenyl alcohol, C2-C6 alkynyl alcohol, C1-C6 alkyl salicylate, aryl alcohol and heteroaryl alcohol, wherein R₁ is H- or a protecting group. In one embodiment the alcohol is methanol.

The present invention is also directed to one or more cosmetic compositions comprising up to 75% by weight of at least one cyrrhetinic alkyl ester of formula (I) or their cosmetically acceptable salt thereof. In an embodiment the cyrrhetinic alkyl ester compounds of the invention are present in the cosmetic composition in an amount from about 0.001% to about 75% by weight of the cosmetic composition. The cosmetic composition may also contain one or more cosmetically acceptable carriers. Such carriers are well known.

For the purposes of the present invention, the use of the term “cosmetic composition” is understood to mean a composition suitable for application to the human body. A cosmetic composition is typically applied to the body for beautifying, cleansing, moisturizing or otherwise treating the external surface of the body, including by cleansing, coloring, conditioning, or protecting the external surface of the body part such as, for example, the skin, nails, lips, or hair. Examples of cosmetic compositions in which the present cyrrhetinic alkyl ester compounds can be used includes skin moisturizers, sunscreens, self-tanning compositions, after-sun care compositions, makeup, protein concentrates, anti-wrinkle or anti-aging compositions, skin firming compositions, skin lightening composition, topically applied therapeutic compositions, hair care compositions, shaving preparation compositions, depilatory compositions, and cleansers.

The present invention is also directed to methods of treating inflammation comprising administering to a host in need of such treatment a therapeutically effective amount of at least one of the compounds of the present invention.

### DETAILED DESCRIPTION OF THE INVENTION

The present invention is directed to cyrrhetinic alkyl ester compounds having the formula (I) and their cosmetically acceptable salts thereof. As previously indicated the compounds of the invention include those of formula (I) or their cosmetically acceptable salts thereof where R₂ is a substituted or unsubstituted moiety selected from the group consisting of C1 to C6 alkyl, C2 to C6 alkenyl, C2 to C6 alkynyl,

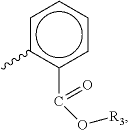

aryl and heteroaryl, wherein R₃ is C1 to C6 alkyl. In a particularly suitable embodiment R₂ may be a methyl group. In another particularly suitable embodiment R₂ may be a moiety formed by the esterification of methyl salicylate at the 2-hydroxy position. In addition, as previously indicated, R₁ may be hydrogen (H-) or an alcohol protecting group, with particularly suitable embodiments including R₁ as hydrogen or an acetyl group.

Exemplary substituents of the R₂ moiety include, without limitation, hydroxyl, amino, alkoxy, alkylthio, alkylamino, arylalkyl, alkylaryl, aryl, heteroaryl, heterocycloalkyl, halogen, alkylsufinyl and alkylsulfonyl. As used herein, alkyl is a straight or branched C1-C6 saturated hydrocarbon chain; alkenyl is a straight or branched C2 to C6 unsaturated hydrocarbon chain having a single double bond; alkynyl is a straight or branched C2 to C6 unsaturated hydrocarbon chain having a single triple bond; alkoxy is a group having an oxygen atom with an alkyl group bonded thereto; aryl is a 6 carbon aromatic ring that may be substituted with 1 to 3 alkyl, hydroxyl or amino substituents; heteroaryl is an aromatic 6 membered carbon ring having as ring members one to three independently selected atoms of nitrogen, oxygen and sulfur; heterocycloalkyl is a saturated 5 to 6 membered carbon ring having as ring members one to three independently selected atoms of nitrogen, oxygen and sulfur; and halogen may be selected from a Cl, Br or F moiety.

The present invention also includes a method of making the cyrrhetinic alkyl ester compounds having the formula (I) and cosmetically acceptable salts thereof, the method comprising the step of esterifying glycyrrhetinic acid of formula (II) with a substituted or unsubstituted alcohol selected from the group consisting of C1-C6 alkyl alcohol, C2-C6 alkenyl alcohol, C2-C6 alkynyl alcohol, C1-C6 alkyl salicylate, aryl alcohol or heteroaryl alcohol. Methods of esterifying acids with alcohols to make alkyl esters are well known to those skilled in the art.

In a first embodiment of the method of making the cyrrhetinic alkyl ester compounds of the invention, the step of esterifying comprises reacting glycyrrhetinic acid of formula (II) in the presence of a substituted or unsubstituted alcohol, e.g., a C1-C6 alcohol, such as methanol and an acid. Exemplary acids include sulfuric acid and carboxylic acids. The mixture may be refluxed to produce the cyrrhetinic alkyl ester compounds of formula (I). An exemplary scheme of this first embodiment of the method of the invention is shown in scheme (I) below.

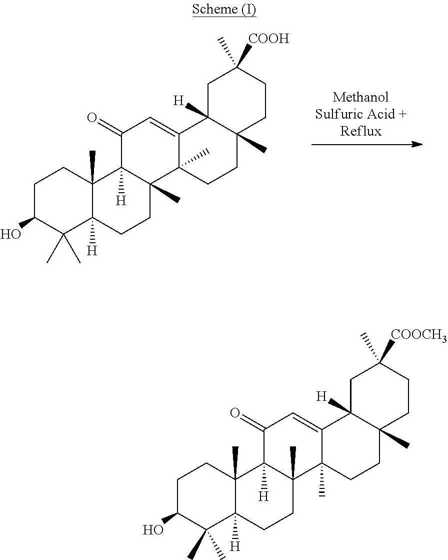

A second embodiment of the method of esterifying the cyrrhetinic acid of formula (II) to make the substituted cyrrhetinic alkyl ester compounds of the invention having the formula (I) includes reacting the cyrrhetinic acid compound in the presence of a substituted or unsubstituted alcohol, e.g., a C1-C6 alkyl alcohol, such as methanol and a coupling agent. Exemplary coupling agents include N,N′-Dicyclohexylcarbodiimide (“DCC”), 1-Ethyl-3-(3-dimethylaminopropyl)carbodiimide (“EDAC”), and N,N′-diisopropylcarbodiimide (“DIC”) . In this embodiment, the reaction generally takes place in an organic solvent. Exemplary reaction solvents include ethylene dichloride, THF and dimethyl amino pyridine and mixtures thereof. An exemplary scheme of this second embodiment of the method of this invention is shown in scheme (II) below.

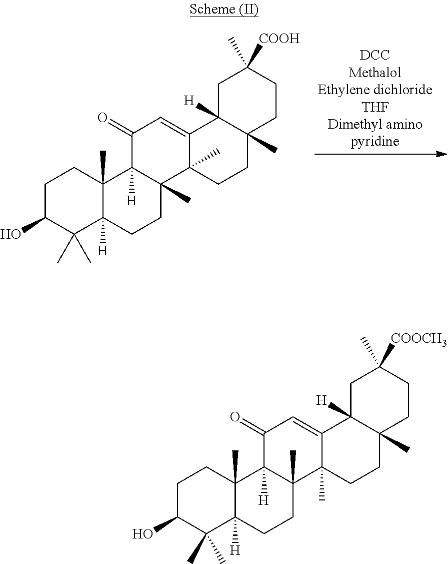

A third embodiment of the method of esterifying the cyrrhetinic acid of formula (II) to make the cyrrhetinic alkyl ester compounds of the invention having the formula (I) includes reacting the cyrrhetinic acid compound in the presence of a substituted or unsubstituted C1-C6 alkyl salicylate such as methyl salicylate and a coupling agent. Exemplary coupling agents include 1-Ethyl-3-(3-dimethylaminopropyl)carbodiimide (“EDAC”), N,N′-Dicyclohexylcarbodiimide (“DCC”) or N,N′-diisopropylcarbodiimide (“DIC”) . The reaction may also include a nucleophilic agent, such as DMAP and a tertiary amine base, such as DIEA. In this embodiment, the reaction generally takes place in an organic solvent, such as THF. An exemplary scheme of this third embodiment of the method of this invention is shown in the scheme (III) below.

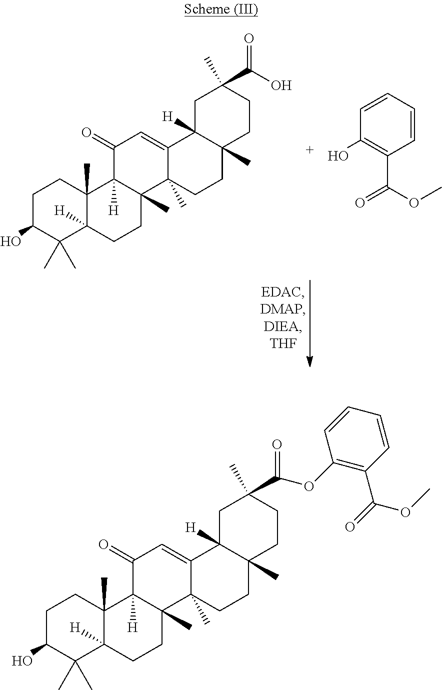

The method of making the compound of formula (I) or the cosmetically acceptable salts thereof may include an optional initial protecting step before the step of esterification. Examples of such a protecting step incorporated into the first and second embodiments of the methods illustrated herein is shown below in schemes (I)(a) and (II)(a), using an acetyl group as the protecting group.

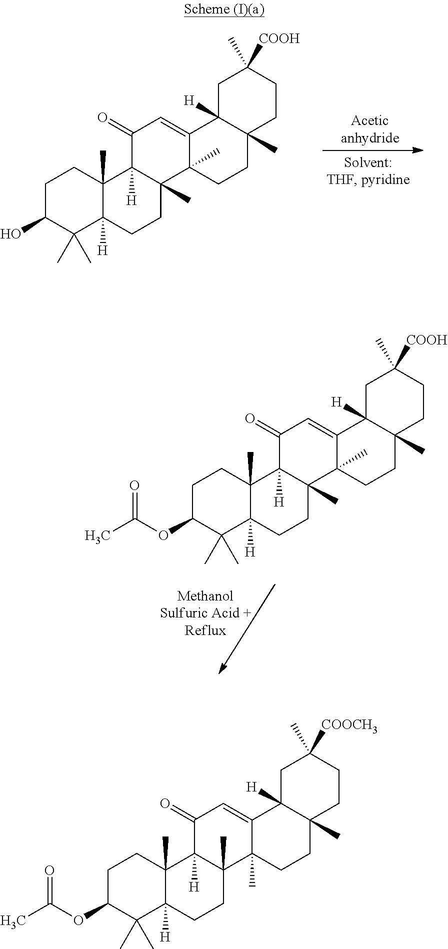

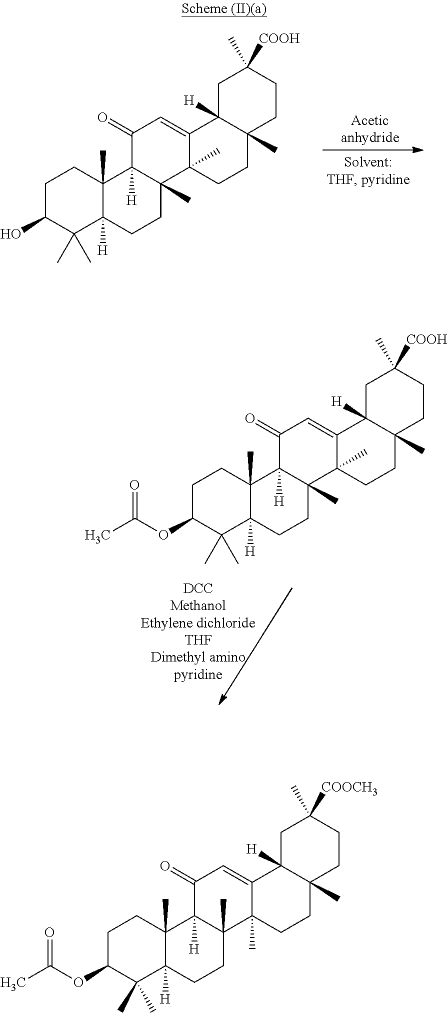

The method of making the compound of formula (I) or the cosmetically acceptable salts thereof may also include an optional deprotecting step, an example of which is shown in scheme (IV) below.

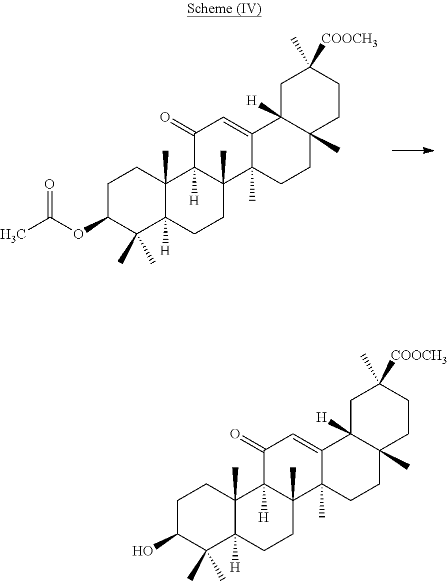

Each of the reaction products for schemes (I), (I)(a), (II), (II)(a), (III) and (IV) may be purified or separated using standard procedures, and cosmetically acceptable salts prepared thereof as desired.

The cyrrhetinic alkyl ester compounds of the present invention possess many advantageous properties for the skin and may be useful for the treatment of various skin conditions. For example, the cyrrhetinic alkyl ester compounds may be useful for the treatment of mild or moderate form of dry skin as it is believed to have moisturizing and/or skin-softening properties. Accordingly, the cyrrhetinic alkyl ester compounds may prevent or cure the occurrence of any cracking, flaking, scaling, or chapping of the skin caused by dry skin. In addition, the cyrrhetinic alkyl ester compounds may be used to prevent, cure, or ameliorate acne, psoriasis, seborrhea, keratosis, diaper rash, sunburn, and windburn.

The cyrrhetinic alkyl ester compounds of the invention may also be useful for the treatment, preventively or curatively, of wrinkles and/or fine lines, wizened skin, a lack of elasticity and/or of tonus of the skin, thinning of the dermis, the degradation of collagen fibers, flaccid skin, thinned skin, and the internal degradation of the skin following exposure to ultraviolet radiation.

It is believed that the cyrrhetinic alkyl ester compounds of the present invention is also advantageous due to the ease of manufacture and handling thereof.

It is also believed that the cyrrhetinic alkyl ester compounds of the present invention do not rapidly degrade, either in storage or formulation, resulting in a loss of activity and/or a change in color. Therefore, the cyrrhetinic alkyl ester compounds are expected to be stable and have a long shelf-life. The cyrrhetinic alkyl ester compounds of the present invention will have an increased penetration and permeability therefore it is more significantly absorbed by the skin to improve the condition and appearance thereof.

The cyrrhetinic alkyl ester compounds may also be useful as an anti-inflammatory treatment, as they have been shown to reduce the secretion of IL-8 in normal human epidermal keratinocytes (NHEK). This result is unexpected as the cyrrhetinic compound of the present invention is formed from the glycyrrhetinic acid of formula (II), a compound that is toxic when applied topically and does not have anti-inflammatory properties.

The present invention is also directed to methods of treating inflammation comprising administering to a host in need of such treatment a therapeutically effective amount of at least one of the compounds of the present invention.

In one embodiment of the method of treating inflammation, the route of administration is topical. Such topical application may be daily, twice daily, thrice daily, or as needed. In another embodiment of the method of treating inflammation, the compound of the present invention is in the form of a cosmetic composition.

The present invention is also directed to one or more cosmetic compositions comprising up to 75% by weight of at least one of the cyrrhetinic alkyl ester compounds of formula (I) or cosmetically acceptable salts thereof. In an embodiment, the cyrrhetinic alkyl ester compounds of the invention are present in the cosmetic composition in an amount from about 0.001% to about 75% by weight of the cosmetic composition.

The cosmetic compositions of the present invention, which include at least one or more of the compounds of the present invention, are useful for a variety of cosmetic purposes due to the properties mentioned above with regard to the cyrrhetinic alkyl ester compounds.

In addition to the compounds of the present invention, the cosmetic compositions of the present invention include a carrier. The carrier for use in formulating the cosmetic compositions may comprise one or more compounds which is selected based on the particular intended use of the composition. The carrier may be inorganic or organic in nature; it must be non-toxic and non-irritating. The carrier must also be compatible with the cyrrhetinic alkyl ester compound used in the composition.

Based on the intended use, the compositions may be care, treatment, cleansing, and/or protective products for facial or body skin; anti-wrinkle or anti-aging compositions; skin firming compositions; skin lightening compositions; compositions for irritated skin; sunscreen compositions, artificial tanning (self-tanning) compositions or after-sun care compositions; hair care and/or scalp care compositions; shaving preparation compositions; depilatory compositions; or make-up products for the skin of the face or body.

The cosmetic compositions of the present invention may also include one or more optional ingredients. Examples of the optional ingredients include but are not limited to lubricants, preservatives, perfumes, and colorants. The optional ingredients should be chemically inert with respect to each other, and with respect to the cyrrhetinic alkyl ester compound(s) used in the composition.

The cosmetic compositions of the present invention may be prepared and used in the form of a lotion, cream, ointment, stick, soap, or other forms commonly employed in the art of cosmetic and skin care formulation. The compositions may be in an emulsion form.

For example, for use in treating dry skin, the cosmetic composition of the present invention may be prepared employing an effective amount (up to about 75 wt %) of the cyrrhetinic alkyl ester compound of the present invention in a cosmetically acceptable carrier, e.g., a hydrophilic ointment or petrolatum. In a preferred embodiment, an amount of about 1 to about 20 wt % of the cyrrhetinic alkyl ester compound is employed. In a more preferred embodiment, an amount of about 5 to about 15 wt % of the cyrrhetinic alkyl ester compound is employed. It should be understood that smaller amounts of the cyrrhetinic alkyl ester compound may be used in the cosmetic composition, for example, when the compound is used together with one or more other skin moisturizer or softener, or when the compound is used in cosmetic compositions that are designed primarily for other types of cosmetic functions. Examples of other skin moisturizers or softeners may include an alkoxyalkylamide compound, a ceramidyl glycyrrhizate compound and a glycyrrhetinyl glycyrrhizate compound.

When an aqueous carrier is used, the cosmetic composition may comprise about 10% to about 90% by weight of water. Preferably, the composition comprises about 10% to about 65% by weight of water. More preferably, about 10% to about 40% by weight of water, and even more preferably, about 15% to about 30% by weight of water. It should be understood however, that the water in the composition can be totally or partly eliminated by the use of non-aqueous or partially aqueous carriers.

A general non-limiting example of an aqueous composition according to the present invention is as follows:

about 0.1 to about 7 wt % emulsifying agent(s)

about 0.1 to about 15 wt % emollient(s)

about 0.1 to about 15 wt % compound of the present invention

about 0.1 to about 5 wt % lubricant(s)

about 0.1 to about 1 wt % preservative(s)

about 0.1 to about 1 wt % perfume(s)

about 0.01 to about 30 wt % colorant(s)

water to make up to 100 wt %.

Lists of carriers and optional ingredients, which are well known in the art, are disclosed, for example, in “Cosmetics: Science and Technology,” edited by M. S. Balsam and E. Sagarin, 2nd Edition, 1972, Wiley Pub. Co.; “The Chemistry and Manufacture of Cosmetics” by M. G. DeNavasse; and “Harry's Cosmeticology,” J. B. Wilkinson et al., 7th Edition, 1982, Chem. Pub. Co.; the disclosures of each of the above being incorporated herein by reference.

The cyrrhetinic alkyl ester compounds of the present invention may be topically applied in uncompounded form to the areas of the skin to be treated therewith. Whether used as is, or in a compounded or compositional form, the cyrrhetinic alkyl ester compounds of the present invention may be topically applied one or more times per day to the area of the skin to be treated for a period of time, e.g., about 7 to 30 days, in order to achieve the desired effect. More preferably, the cyrrhetinic alkyl ester compounds of the present invention may be applied about 1 to 2 times per day.

A makeup composition, for example, may incorporate the alkyl cyrrhetinic alkyl ester compound of the present invention in a small amount, generally about 0.001 to about 10 wt %, preferably about 0.02 to about 1.0 wt % of the composition. In addition, the makeup composition may comprise about 1 to about 40 wt %, preferably about 10 to about 20 wt %, of a coloring agent in a suitable carrier. Suitable coloring agents include inorganic and organic pigments which are usable in cosmetic formulations. Examples of these pigments include carmine, bismuth oxychloride, zinc oxide, ferric oxide, ferrous oxide, kaolin, ultramarine violet, ultramarine blue, chromium oxide, chromium hydroxide, silica, manganese violet, talc, mica, and titanium oxide. The examples also include lakes of organic colorants such as FD&C Red No. 7 calcium lake, FD&C Yellow No. 5 aluminum lake, FD&C Red No. 9 barium lake, carbon black, and FD&C Red No. 30.

As used herein, “about” or “approximately” generally means within 20 percent, preferably within 10 percent, and more preferably within 5 percent of a given value or range.

Various tests may be carried out to compare formulations containing the cyrrhetinic alkyl ester compound(s) of the present invention with formulations without a compound of the invention to show the advantageous properties of the cyrrhetinic alkyl ester compounds. Examples of tests are provided below:

| Property | Test(s) |
|---|---|
| Anti-inflammatory effect | Phorbol 12-myristate 13-acetate (PMA) |
| | stimulation |
| Collagen synthesis stimulation | Human fibroblasts culture |
| Melanin formation inhibition | Mushroom tyrosinase; B16 melanoma |
| | cell culture |
| Anti-oxidant effect | Lipid peroxidation |
| Matrix Metalloproteinases | Fibroblast cell culture |
| (MMPs) inhibition | |
| Percutaneous absorption | Tape stripping; Bronaugh cell diffusion |
| Hydration | Dermal phase meter |

The example set forth below show methods of the use of a cyrrhetinic alkyl ester compound of the present invention, cyrrhetinic methyl ester, i.e., the compound of formula (I) where R₁ is H- and R₂ is a methyl group, in a cosmetic composition. The following example is merely illustrative of the scope of the present invention and is not intended as a limitation upon the scope thereof.

### EXAMPLE 1

A cosmetic composition may be prepared using a cyrrhetinic alkyl ester compound of the present invention The formulation, in wt %, is as follows:

| Component | Weight % |
|---|---|
| Cyclopentasiloxane | 25.52 |
| Disteardimonium Hectorite | 1.00 |
| PEG-9 Polydimethylsiloxyethyl Dimethicone | 2.50 |
| Polyglyceryl-3 Diisostearate | 0.50 |
| Trimethylsiloxysilicate | 1.50 |
| Boron Nitride | 1.13 |
| Titanium Dioxide, Alumina, Methicone | 5.50 |
| Titanium Dioxide, Triethoxycaprylylsilane, Alumina, Silica | 5.23 |
| Iron Oxides, Methicone | 1.58 |
| 50/50 D9126I Cangee | 0.94 |
| Iron Oxides, Methicone | 0.12 |
| Mica, Methicone | 0.01 |
| Ethylhexyl Methoxycinnamate | 3.5 |
| Dimethicone, Dimethicone/PEG-10/15 crosspolymer | 4.00 |
| Dimethicone, Dimethicone crosspolymer | 2.00 |
| Phenyl Trimethicone | 1.50 |
| Phenoxyethanol | 0.70 |
| Tocopheryl Acetate | 0.01 |
| Water | 35.649 |
| Sodium Chloride | 0.50 |
| Tetrasodium EDTA | 0.01 |
| Potassium Sorbate | 0.20 |
| Pullan, Sorbitol, Trehalose, Acacia Sengal Gum | 1.25 |
| Glycerin | 3.00 |
| Cyrrhetinic methyl ester | 0.001 |
| Xanthan Gum | 0.20 |
| Laureth-7 | 0.50 |
| Caprylyl Glycol | 0.70 |
| Silica | 0.75 |
| TOTAL | 100.00 |

### EXAMPLE 2

It is believed that the cyrrhetinic alkyl ester compounds of the present invention are useful for reducing inflammation in human skin. In an exemplary test, NHEK were cultured in a medium for 24 hours. The cells were then treated or not (control) with either cyrrhetinic methyl ester or the reference compound (staurosporine) and pre-incubated for 24 hours. Then the culture medium was removed and replaced with another culture medium containing or not (control) the cyrrhetinic methyl ester or the reference compound and containing an inflammatory inducer (PMA) and the cells were incubated for 24 hours. A control without inducer was performed in parallel. After incubation, the culture supernatants were collected in order to measure the quantity of secreted IL-8. The results below show up to a 73% inhibition of IL-8 secretion in the NHEK treated with cyrrhetinic methyl ester.

| | | | % | |
|---|---|---|---|---|
| | | Avg. IL-8 | Stimulated | |
| Compound | Concentration | (pg/ml) | Control | % Inhibition |
| Control | — | <44 | <1 | 100 |
| (no PMA) | | | | |
| Control | — | 3200 | 100 | 0 |
| Staurosporine | 10⁻⁹M | 588 | 18 | 83 |
| Cyrrhetinic | 15.625 μM | 1349 | 42 | 59 |
| methyl ester | 31.25 μM | 907 | 28 | 73 |
| | 62.5 μM | 902 | 28 | 73 |
| Glycyrrhetinic | 0.5 μM | 3037 | 95 | 5 |
| acid | 1 μM | 3470 | 108 | −9 |
| | 2 μM | 4450 | 139 | −40 |

1. A compound of formula (I): 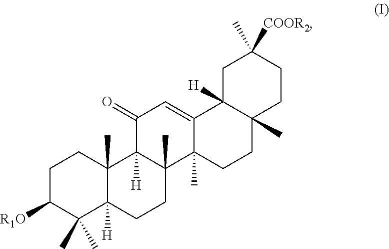
or a cosmetically acceptable salt thereof,
wherein R₁ is selected from the group consisting of H- and a protecting group; and
R₂ is a substituted or unsubstituted moiety selected from the group consisting of C1 to C6 alkyl, C2 to C6 alkenyl, C2 to C6 alkynyl, 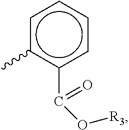
aryl and heteroaryl, wherein R₃ is C1 to C6 alkyl.

2. The compound of claim 1 wherein R₁ is H- and R₂ is a methyl group.

3. The compound of claim 1 wherein R₁ is H- and R₂ is 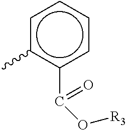
and R₃ is methyl.

4. The compound of claim 1 wherein R₁ is a protecting group selected from the group consisting of acetyl, benzoyl, benzyl, beta-methoxymethyl ether, methoxymethyl ether, p-methoxy benzyl ether, methylthiomethyl ether, pivaloyl, tetrahydropyranal, trityl and silyl ether .

5. The compound of claim 1, wherein R₁ is a protecting group that is an acetyl group and R₂ is a methyl group.

6. A cosmetic composition comprising up to 75% by weight of the compound of claim 1.

7. The cosmetic composition of claim 6, wherein R₁ is H- and R₂ is a methyl group.

8. The cosmetic composition of claim 7, further comprising a cosmetically acceptable carrier and wherein the compound is in an amount of about 0.001% to about 75% by weight of the composition.

9. The cosmetic composition of claim 6, wherein R₁ is H-, R₂ and R₃ is methyl.

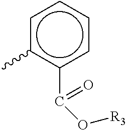

10. The cosmetic composition of claim 9, further comprising a cosmetically acceptable carrier and wherein the compound is in an amount of about 0.001% to about 75% by weight of the composition.

11. A method of making the compound of formula (I): 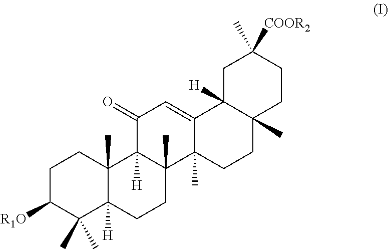
or a cosmetically acceptable salt thereof,
wherein R₁ is selected from the group consisting of H-and a protecting group; and
R₂ is a substituted or unsubstituted moiety selected from the group consisting of C1 to C6 alkyl, C2 to C6 alkenyl, C2 to C6 alkynyl, 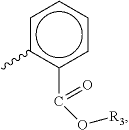
aryl and heteroaryl, wherein R₃ is C1 to C6 alkyl, the method comprising the step of:
reacting a substituted or unsubstituted alcohol selected from the group consisting of C1 to C6 alkyl alcohol, C2 to C6 alkenyl alcohol, C2 to C6 alkynyl alcohol, C1-C6 alkyl salicylate, aryl alcohol or heteroaryl alcohol with glycyrrhetinic acid compound of formula (III): 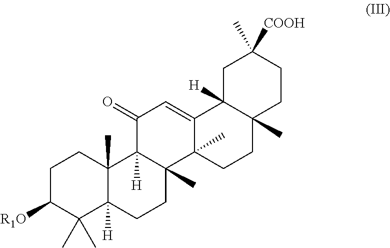
wherein R₁ is H- or a protecting group, to form the compound of formula (I) or cosmetically acceptable salt thereof.

12. The method of claim 11, wherein the step of reacting further comprises the addition of an acid to the mixture and a step of refluxing the mixture.

13. The method of claim 12, wherein the acid is sulfuric acid.

14. The method of claim 13, wherein the alcohol is methanol.

15. The method of claim 11, wherein the step of reacting further comprises adding a coupling agent to the reaction mixture.

16. The method of claim 15, wherein the coupling agent is N,N′-dicyclohexyl carbodiimide.

17. The method of claim 11, further comprising prior to the reacting step, a step of protecting glycyrrhetinic acid of formula (II): 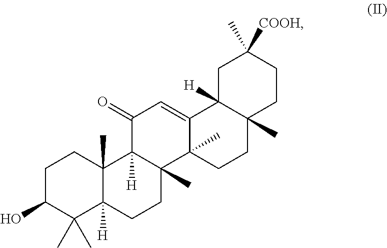
to form the protected glycyrrhetinic acid compound of formula (III); 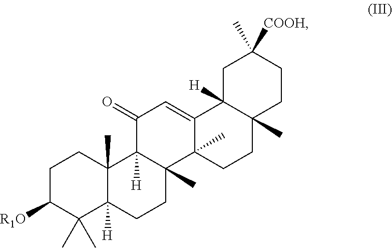
wherein R₁ is a protecting group.

18. The method of claim 17, wherein R₁ is an acetyl group.

19. The method of anyone of claim[[s]] 17 and 18, further comprising a deprotecting step to form the compound of formula (I) wherein R₁ is H-.

20. A method of treating inflammation comprising administering to a host in need of such treatment a therapeutically effective amount of the compound of claim 1.

21. The method of claim 20 wherein the route of administration is topical.

22. The method of claim 20 wherein the compound is in the form of a cosmetic composition.
